(19) United States
(12) Patent Application Publication
(10) Pub. No.: US 2023/0060091 A1
(43) Pub. Date: Feb. 23, 2023

(54) MYCOTOXIN REMOVAL FROM THE BUILT ENVIRONMENT

(71) Applicants: Robert John GOLDSWORTHY, Newcastle, New South Wales (AU); Robert Adam GOLDSWORTHY, Newcastle, New South Wales (AU)

(72) Inventors: Robert John GOLDSWORTHY, Newcastle (AU); Robert Adam GOLDSWORTHY, Newcastle (AU)

(21) Appl. No.: 17/790,090

(22) PCT Filed: Dec. 31, 2020
(86) PCT No.: PCT/AU2020/051446

§ 371 Date: Jun. 29, 2022

(30) Foreign Application Priority Data
Jan. 3, 2020 (AU) 2020200049
Jan. 3, 2020 (AU) 2020902578
Nov. 30, 2020 (AU) 2020904429

(51) Int. Cl. B08B 3/08; F24F 8/158; F24F 8/108; A61L 9/014; A61L 2/18; B08B 1/00; B08B 3/02; B01D 46/62; B01D 39/20

(52) U.S. Cl. CPC B08B 3/08; F24F 8/158; F24F 8/108; A61L 9/014; A61L 2/18; B08B 1/006; B08B 3/02; B01D 46/62; B01D 39/2055; A61L 2209/14; A61L 2209/22; A61L 2202/17; A61L 2101/06

(57) ABSTRACT

A method for the removal of mycotoxins from a space associated with a built environment is disclosed. The method may include one or more of the steps of: filtering air within the space using a first filter including a High-Efficiency Particulate Air (HEPA) filter and a second filter including a carbon filter; and, applying a cleaning product including a high alkaline product to one or more surfaces proximate the space.

### RELATED APPLICATIONS

This application claims priority from Australian provisional patent application nos. 2020902578, 2020200049 filed on 3 Jan. 2020 and Australian provisional patent application 2020904429 filed on 30 Nov. 2020, the contents of which are incorporated by reference.

### TECHNICAL FIELD

The invention relates to a method for the removal fungi and fungal produced mycotoxins from the built environment.

### BACKGROUND

Fungus such as mould may produce chemical by-products known as mycotoxins. Such mycotoxins may be hazardous to human health in particular when such mycotoxins are present as a result of mould within the built environment such as within a person's home.

Accordingly, there is a need for the removal of fungal growth from the built environment. Such removal may include removal of the active growth by physical means either by cleaning or by removal of the building materials. In addition, air scrubbing either mechanical, or non-mechanical, may be utilised.

A problem with removal via such physical means or air scrubbing is that whilst the fungal growth may be reduced or generally removed, the associated mycotoxins may not be adequately removed or may even remain.

The invention disclosed herein seeks to overcome one or more of the above identified problems or at least provide a useful alternative.

### SUMMARY

A method for the removal of mycotoxins associated with fungus from the built environment is disclosed. The method generally seeks to reduce or eliminate the further generation of mycotoxins during the removal process as it has been discovered that physical means to remove the fungus, such as, but not limited to, scrubbing were actually increasing and releasing mycotoxins, and it also has been discovered that filtration means may only capture the fungal spores with at least some or all mycotoxins passing through and being circulated by the exhaust of the filter.

In more detail, in accordance with a first main aspect, a method for the removal of mycotoxins from a space associated with a built environment is provided. The method may include one or more of the steps of: filtering air within the space using a first filter including a High-Efficiency Particulate Air (HEPA) filter and a second filter including a carbon filter; and, applying a cleaning product including a high alkaline product to one or more surfaces proximate the space.

In an aspect, the step of filtering air includes operation of a filtration unit including the HEPA filter and the carbon filter fitted downstream of the HEPA filter.

In another aspect, the carbon filter includes or is one of an activated carbon filter and a paper carbon filter.

In yet another aspect, the step of filtering air includes a third filter, the third filter being a pre-filter arrangeable upstream of the HEPA filter.

In yet another aspect, the high alkaline product is a product with a pH in the range of about 7 to 14.

In yet another aspect, the cleaning product is or includes Sodium Hypochlorite (NaClO).

In yet another aspect, the high alkaline product is a high alkaline additive being or including a surfactant.

In yet another aspect, the step of applying the cleaning product includes wiping the one or more surfaces.

In yet another aspect, the wiping includes use of a cloth.

In yet another aspect, the cloth is a microfibre cloth.

In yet another aspect, the step of filtering includes placing a filter unit central to an affected area of the space.

In yet another aspect, the step applying the cleaning product includes at least two stages, first stage including removing visible mould staining from hard surfaces only by wiping with a first applicator with the cleaning product, and the second stage includes a second wiping of the hard surfaces with a second applicator including a second cleaning product.

In yet another aspect, one or both of the first and second applicators are or include microfibre cloths.

In yet another aspect, the cleaning product is Sodium Hypochlorite (NaClO) containing a high alkaline additive.

In yet another aspect, the second cleaning product is water including a high alkaline additive.

In yet another aspect, the first and second applicators are replaced after about cleaning 2 m² of the hard surfaces.

In yet another aspect, wherein the step applying the cleaning product includes a third stage of applying a third cleaning product to soft surfaces proximate the space.

In yet another aspect, the third stage includes spraying the soft surfaces with the third product including a high alkaline additive.

In yet another aspect, the third stage includes steam cleaning after spraying the soft surfaces.

In yet another aspect, the space includes an air conditioner inlet or outlet and a filtering unit is placed proximate the inlet or outlet.

### BRIEF DESCRIPTION OF THE FIGURES

The invention is described, by way of non-limiting example only, by reference to the accompanying figures, in which;

FIG. 1 is a first example method of removal of mycotoxin from the built environment; and

FIG. 2 is a second example method of removal of mycotoxin from the built environment

### DETAILED DESCRIPTION

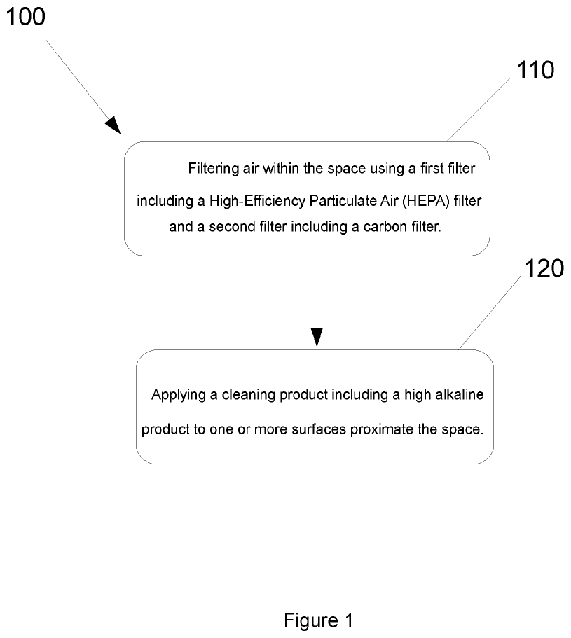

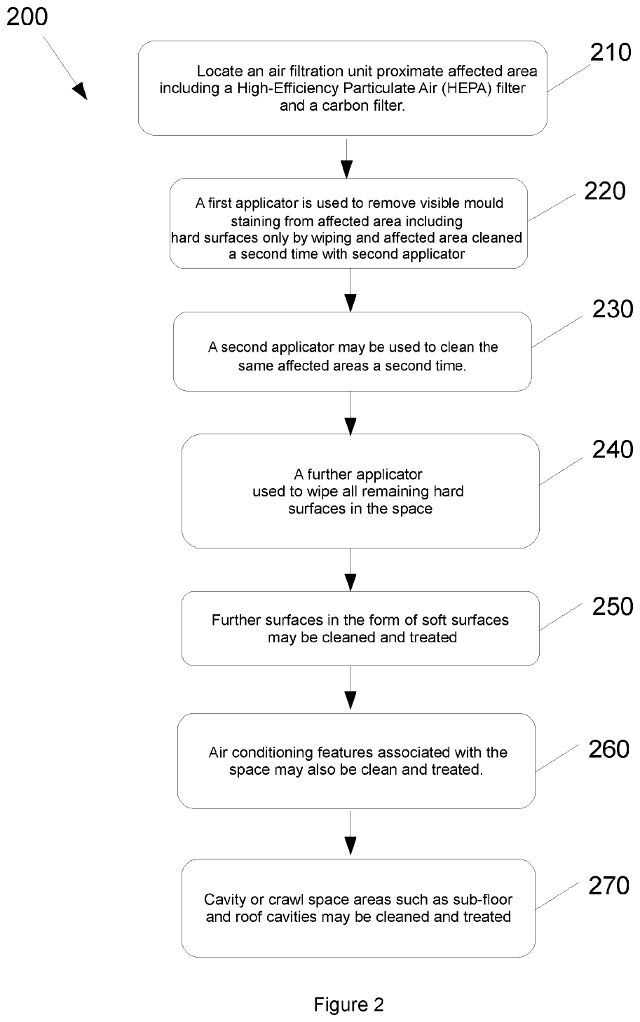

Referring to FIGS. 1 and 2, there are disclosed two examples of a method of removal of mycotoxins from the built environment. The method has been developed as a result of the surprising discovery that existing methods, that may include mechanical cleaning such as scrubbing and air filtration, to clean and remove fungus such as mould did not address the removal of associated mycotoxins. The method 100, as shown in FIG. 1, includes two main steps and may include one or more further steps as described in relation to method 200 with reference to FIG. 2 below.

Each of the methods 100, 200 may be used within and associated with the built environment including, but not limited to houses, units and commercial premises. Persons carrying out the methods may wear personal protective equipment. Such personal protective equipment may include: full disposable protective suit, or coveralls, with hood and boot coverings rated to withstand airborne particulates and chemicals; gloves to protect skin, full face respirator, fitted with filters rated for protection against particulates and chemicals, minimum P3.

Referring to FIG. 1, the method 100 for the removal of mycotoxins from a space associated with a built environment is shown. The space may be a room, a hallway, an underfloor area or the like. There may be one or more affected areas within the space that include surfaces such as walls and carpet that exhibit signs of fungal growth such as mould.

The method 100 includes, at step 110, filtering air within the space using a first filter including a High-Efficiency Particulate Air (HEPA) filter and a second filter including a carbon filter. The step of filtering air may include operation of a filtration unit (not shown) including the HEPA filter and the carbon filter fitted downstream of the HEPA filter. A third filter in the form of a prefilter may also be fitted upstream of the HEPA filter. The carbon filter may be activated carbon filter or a paper carbon filter. The carbon-based filter assists to capture mycotoxins that pass through the HEPA filter. The filtration unit may be placed at or proximate the affected area within the space.

The method 100, at step 120, may further include applying a cleaning product including a high alkaline product to one or more surfaces proximate the space. The clean product may be applied at the same time as the filtering of the air. In particular, the application may include use of one or more applicators. The applicators may be cloths, in particular, microfibre cloths and the application may include only wiping of surfaces.

The cleaning product may be or include Sodium Hypochlorite (NaClO) and the high alkaline product is a product with a pH in the range of about 7 to 14. An example of a filter unit may include a barrel filter having an electrostatically charged high efficiency pre-filter, a HEPA filter that has an efficiency of 99.97% at 0.3 microns and a final stage filter being a 6 kg activated carbon shell filter. Other types of filters including square filters or similar may also be used. The carbon filter may be an activated carbon filter of a thickness between 1-4 cm and being the same dimensions as that of the HEPA filter to ensure that all of the air passing through the HEPA filter also passes through the activated carbon filter.

It has been found that use of the high alkaline cleaning product in combination with wiping, as opposed to hard mechanical scrubbing, is less disruptive to release mycotoxins and allows for removal of mycotoxins. Further, any mycotoxins released are captured by the filtration unit. The method 100 may include further steps such as one or more the steps as described in relation to the second example method 200 below.

Turning now to method 200, a more specific example of mycotoxin removal is provided. Like the first method, in this method all workers or persons present during the below method are to be wear and fitted with personal protective equipment as set out above. The method 200 may include one or more of the below steps.

At step 210, the method includes placing or locates an air filtration unit, scrubber, or air purifier with both HEPA (high-efficiency particulate air) and activated carbon or paper carbon filters central to affected area. The activated carbon or paper carbon filters should be placed within the air filtration unit in a position that is after or downstream of the HEPA filter and before the clean air outlet. It is noted that the use of negative air pressure, and negative HEPA and activated carbon or carbon filtered air pressure can aid in the removal of airborne mycotoxins via extraction of the contaminated air. In addition, the use of positive air pressure, and positive HEPA and activated carbon or carbon filtered air pressure can aid in the removal of airborne mycotoxins via dilution of the indoor air. The filtering may be continued for the duration of the steps below until completion.

At step 220, a first applicator is used to remove visible mould staining from affected area including hard surfaces only by wiping. The applicator may be a microfibre fibre cloth dampened with a cleaning product which may be Sodium Hypochlorite (NaClO) containing a high alkaline additive. It is noted that the use of a bleach product with the high alkaline additive is only a necessary step to remove visible mould. If there is no visible mould, this step would be unnecessary.

At step 230, a second applicator may be used to clean the same affected areas a second time. The second applicator may be a cloth, in particular, a new microfiber cloth dampened with H2O containing a high alkaline additive.

The user should ensure that cloths are rinsed or replaced approximately every 2 square meters and used cloths may be placed in a bag such as a Ziplock bag for appropriate cleaning or disposal.

At step 240, the user may use a further applicator, such as a cloth, to wipe all remaining hard surfaces in the space. The further applicator may be a microfiber cloth dampened with water and high alkaline product. The cloths are to be rinsed or replaced as specified above.

At step 250, further surfaces in the form of soft surfaces may be cleaned and this may include pre-spraying all carpets and soft furnishings with water and high alkaline product and, then steam cleaning using water only. Similarly, all hard flooring surfaces may also be wiped with microfiber cloth dampened with H2O and high alkaline solution. Hard floorings may also be steam cleaned following pre-spray with the high alkaline solution.

At step 260, air conditioning features associated with the space may also be cleaned and treated. This may include cleaning air conditioner and/or HVAC filters initially by running under a faucet and then spraying the filters with water and high alkaline solution. During cleaning of the conditioning features, the air filtration unit as described above may be placed close to the air outlet being cleaned. The barrel and accessible parts of air conditioned and/or HVAC unit are then cleaned using water and high alkaline solution. The filters are then replaced the air conditioner and/or HVAC is then operated for a time period, such as at least 5 minutes whilst the air filtration unit is still running.

At step 270, cavity or crawl space areas such as sub-floor and roof cavities may be cleaned and treated to remove mycotoxins. During this step, the air filtration unit may be moved into cavity or crawl space, or air separate air movement may be used, and arranged to vent air to outside. Then surfaces of the cavity or crawl space areas may be cleaned using yet a further applicator to wipe all visible mould staining from hard surfaces, ie: frames and linings, using Sodium Hypochlorite containing a high alkaline additive. The applicator may be a microfibre cloth.

It is noted that persons who are not equipped with Personal Protective Equipment (PPE) must be excluded from within the working space for a period to be determined by a calculation based on equipment as shown below.

A÷B=C
A=M3 of the room/area to be treated
B=M3 per hour capacity of air purifier
C=Minimum hours of exclusion from area/room being treated.

Advantageous, there has been described a method for removal of mycotoxins from the built environment that, firstly, addresses the surprising finding that existing filters do not adequately remove or capture mycotoxins, and also the surprising finding that typical cleaning products and methods actually may release more mycotoxins. The described method for removal of mycotoxins utilises a filter with both a HEPA filter and a carbon type filter to specifically capture mycotoxins at the site of an affected area and then includes generally soft cleaning actions including wiping or rubbing with a soft applicator such as cloth, in particular a microfiber cloth, using highly alkaline cleaning products that has been found to remove and capture mycotoxins.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any known matter or any prior publication is not, and should not be taken to be, an acknowledgment or admission or suggestion that the known matter or prior art publication forms part of the common general knowledge in the field to which this specification relates.

While specific examples of the invention have been described, it will be understood that the invention extends to alternative combinations of the features disclosed or evident from the disclosure provided herein.

Many and various modifications will be apparent to those skilled in the art without departing from the scope of the invention disclosed or evident from the disclosure provided herein.

1. A method to remove mycotoxins from a space associated with a built environment, the method including the steps of:
Filtering air within the space using a first filter including a High-Efficiency Particulate Air (HEPA) filter and a second filter including a carbon filter, and at about the same time;
Applying one or more cleaning products in at least two stages to one or more surfaces proximate the space including wiping the one or more surfaces, wherein a first stage of the at least two stages includes removing visible mould staining from a hard surface of the more or more surfaces by wiping the hard surface using a first applicator with a first cleaning product of the one or more cleaning products that includes a high alkaline additive, and the second stage of the at least two stages includes a further wiping of the hard surface using a second applicator with a second cleaning product of the one or more cleaning products.

2. The method according to claim 1, wherein the step of filtering air includes operation of a filtration unit including the HEPA filter and the carbon filter fitted downstream of the HEPA filter.

3. The method according to claim 1, wherein the carbon filter includes or is one of an activated carbon filter and a paper carbon filter.

4. The method according to claim 1, wherein the step of filtering air includes a third filter, the third filter being a pre-filter arrangeable upstream of the HEPA filter.

5. (canceled)

6. The method according to claim 1, wherein the one or more cleaning products is or includes Sodium Hypochlorite (NaClO).

7. The method according to claim 1, wherein the high alkaline product is a high alkaline additive.

8. (canceled)

9. The method according to claim 1, wherein the wiping includes use of a cloth.

10. The method according to claim 9, wherein the cloth is a microfibre cloth.

11. The method according to claim 1, wherein the step of filtering includes placing a filter unit central to an affected area of the space.

12. (canceled)

13. The method according to claim 1, wherein one or both of the first and second applicators are or include microfibre cloths.

14. The method according to claim 1, wherein the one or more cleaning products is Sodium Hypochlorite (NaClO) containing a high alkaline additive.

15. The method according to claim 1, wherein the second cleaning product is water including a high alkaline additive.

16. The method according to claim 1, wherein the first and second applicators are replaced after about cleaning 2 m² of the hard surfaces.

17. The method according to claim 1, the wherein the step applying the one or more cleaning products includes a third stage of applying a third cleaning product to soft surfaces proximate the space.

18. The method according to claim 17, wherein the third stage includes spraying the soft surfaces with the third product including a high alkaline additive.

19. The method according to claim 18, wherein the third stage includes steam cleaning after spraying the soft surfaces.

20. The method according to claim 1, wherein the space includes an air conditioner inlet or outlet and a filtering unit is placed proximate the inlet or outlet.
